(19) United States
(12) Patent Application Publication
(10) Pub. No.: US 2015/0377869 A1
(43) Pub. Date: Dec. 31, 2015

(54) PURIFICATION OF NANOPARTICLE-ANTIBODY CONJUGATES

(71) Applicant: Bio-Rad Laboratories, Inc., Hercules, CA (US)

(72) Inventors: Tom Berkelman, Oakland, CA (US); Jiali Liao, San Ramon, CA (US)

(21) Appl. No.: 14/749,022

(22) Filed: Jun. 24, 2015

Related U.S. Application Data
(60) Provisional application No. 62/016,752, filed on Jun. 25, 2014.

(51) Int. Cl. G01N 33/533; C07K 1/34; G01N 33/58; C07K 16/00

(52) U.S. Cl. CPC G01N 33/533; C07K 16/00; C07K 1/34; G01N 33/587; G01N 33/582

(57) ABSTRACT

Methods of purifying antibody-nanoparticle conjugates with size exclusion chromatography are provided.

### CROSS-REFERENCE TO RELATED PATENT APPLICATIONS

The present application claims priority to U.S. Provisional Patent Application No. 62/016,752, filed on Jun. 25, 2014, which is incorporated by reference for all purposes.

### BACKGROUND OF THE INVENTION

P-dot nanoparticles (, e.g., as described in Pub. No. US2012/0282632) are highly fluorescent and are useful reporters when conjugated to antibodies or other proteins. Conjugation reactions between antibodies and nanoparticles are generally carried out using excess antibody in order to assure an optimal conjugation ratio. Following conjugation, the conjugate needs to be separated from the excess free antibody, since unconjugated antibody will compete with the conjugate for target binding. Since free antibodies and nanoparticles are of similar size and density, this presents a challenge.

### BRIEF SUMMARY OF THE INVENTION

Methods of purifying an antibody-nanoparticle conjugate from free antibody are provided. In some embodiments, the method comprises providing a mixture of the antibody-nanoparticle conjugate, the free antibody, a polyalkylene glycol surfactant, and a buffer, wherein the ionic strength of the mixture is at least 50 mM; contacting the mixture to a polysaccharide-based size exclusion medium to separate the antibody-nanoparticle conjugate from the free antibody; and collecting fractions enriched for the antibody-nanoparticle conjugate from the medium, thereby purifying the antibody-nanoparticle conjugate from the free antibody.

In some embodiments, the polysaccharide-based size exclusion medium comprises agarose.

In some embodiments, the buffer comprises phosphate. In some embodiments, the buffer is phosphate buffered saline (PBS). In some embodiments, the PBS is at a concentration of 0.5-2.0 X.

In some embodiments, the antibody is an IgG antibody. In some embodiments, the antibody is a tetrameric IgG antibody.

In some embodiments, the nanoparticle is a polymer dot (p-dot). In some embodiments, the p-dot is 5-100 nm in diameter and is a colloidal semiconducting polymer. In some embodiments, the p-dot is fluorescent.

In some embodiments, the surfactant is Pluronic F-68. In some embodiments, the surfactant in the mixture is at a concentration of 0.02%-1.0%.

In some embodiments, the ionic strength of the mixture is 75-300 mM.

In some embodiments, the mixture further comprises free nanoparticle and the medium separates the free nanoparticle from the conjugate.

In another embodiment, the method comprises providing a mixture of the antibody-nanoparticle conjugate, the free antibody, a polyalkalene glycol surfactant, and a buffer, wherein the ionic strength of the mixture is at least 50 mM; contacting the mixture to nanomembrane filter to separate the antibody-nanoparticle conjugate from the free antibody; and collecting fractions enriched for the antibody-nanoparticle conjugate from the filter, thereby purifying the antibody-nanoparticle conjugate from the free antibody.

In some embodiments, the buffer comprises phosphate. In some embodiments, the buffer is phosphate buffered saline (PBS). In some embodiments, the PBS is at a concentration of 0.5-2.0 X.

In some embodiments, the antibody is an IgG antibody. In some embodiments, the antibody is a tetrameric IgG antibody.

In some embodiments, the nanoparticle is a polymer dot (p-dot). In some embodiments, the p-dot is 5-100 nm in diameter and is a colloidal semiconducting polymer. In some embodiments, the p-dot is fluorescent.

In some embodiments, the surfactant is Pluronic F-68. In some embodiments, the surfactant in the mixture is at a concentration of 0.02%-1.0%.

In some embodiments, the ionic strength of the mixture is 75-300 mM.

In some embodiments, the mixture further comprises free nanoparticle and the medium separates the free nanoparticle from the conjugate.

### DEFINITIONS

“Antibody” refers to an immunoglobulin or fragmentary form thereof. The term may include but is not limited to polyclonal or monoclonal antibodies of the classes IgA, IgD, IgE,

IgG, and IgM, derived from human or other mammalian cell lines, including natural or genetically modified forms such as humanized, human, single-chain, chimeric, synthetic, recombinant, hybrid, mutated, grafted, and in vitro generated antibodies. “Antibody” may also include composite forms including but not limited to fusion proteins containing an immunoglobulin moiety. “Antibody” may also include antibody fragments such as Fab, F(ab′)2, Fv, scFv, Fd, dAb, Fc and other compositions, whether or not they retain antigen-binding function.

### BRIEF DESCRIPTION OF THE DRAWINGS

FIG. 1 is a graph comparing absorbance at 463 nm compared to fraction number. The experiment depicted is purification of a nanoparticle-antibody conjugation sample in low ionic strength 20 mM HEPES-KOH buffer on a 30 cm column of Superose 6.

FIG. 2 is a graph comparing absorbance at 463 nm compared to fraction number. The experiment depicted is purification of a nanoparticle-antibody conjugation sample in 1 X PBS, 0.1% Pluronic F-68 on a 30 cm column of Superose 6.

### DETAILED DESCRIPTION OF THE INVENTION

Antibody-nanoparticle conjugates can be difficult to purify from unconjugated free antibody because the conjugates and free antibodies are of approximately the same size and density, and because nanoparticles can be susceptible to aggregation and precipitation. The inventors have surprisingly discovered a combination of conditions that allows for separation of antibody-nanoparticle conjugates from free antibody. Namely, the inventors have discovered that a high ionic strength buffer comprising a surfactant can be used to maintain the solubility of the conjugates and to generate sufficient separation of the conjugate from free antibody on polysaccharide-based size exclusion media to allow for purification of the conjugates.

It is believed that the purification method can be applied to a wide range of antibody-nanparticle conjugates. For example, in some embodiments, the antibody is an IgA, IgD, IgE, IgG, and IgM antibody. In some embodiments, the antibody is an antigen-binding antibody fragment such as, for example, a Fab, F(ab′)2, or Fv, or a fusion protein comprising such fragments. In some embodiments, the antibody is a single-chain antibody, e.g., a scFv, a fusion of the variable regions of the heavy (VH) and light chains (VL) of one or more antibodies. The antibody can be recombinant or naturally-occurring. The antibody can be human, mouse, rat, rabbit, bovine, goat, camel, or from other antibody-producing species

Nanoparticles are particles in a nanoscale, e.g., from about 1 nm to about 1000. In some embodiments, the particles re between 1-300 nm, 5-80 nm, or 8-60 nm. Many nanoparticles are roughly spherical in shape, which results in a dimension being the radius or diameter of the spherical particle. The hydrodynamic radius or diameter can also be used to define the nanoparticle size.

In some embodiments, the nanoparticle is a fluorescent semiconducting polymer dot. Examples of such pdots are described in, e.g., Wu, C., et al., Chem. Mater. 21:3816-3822 (2009); Rahim, N. A. A., et al., Adv. Mater. 21:3492-3496 (2009), Rong et al., ACS Nano 7(1):376-84 (2013); patent publications US 2013/0266957; WO 2012/054525; and US 2012/0282632. Chromophoric pdots can be generated by collapsing polymer into a stable sub-micron sized particle. The nanoparticles provided herein may be formed by any method known in the art for collapsing polymers, including without limitation, methods relying on precipitation, methods relying on the formation of emulsions (e.g. mini or micro emulsion), and methods relying on condensation.

Nanoparticles can be functionalized as desired to link the nanoparticle to an antibody. Exemplary functionalization of nanoparticles is described in, e.g., US Patent Publication No. 2012/0282632. As an example, a nanoparticle can be functionalized to present one or more carboxylic acid moieties, which in turn can be used via one or more linker to an antibody. The conjugate components (e.g., antibody and nanoparticle) can be linked covalently or non-covalently. An example of a non-covalent linkage is a biotin-streptavidin affinity, where one member of the conjugate is biotinylated and the other member of the conjugate is linked to streptavidin. Other examples of linkage options include, but are not limited to direct coupling of nanoparticles to antibody amines; modification of nanoparticles with maleimide and subsequent linkage to an antibody having an exposed thiol (generated, for example, by treating the antibody with mercaptoethylamine or 2-iminothiolane (Traut's reagent)); modification of nanoparticles with hydrazine and linkage to an antibody with oxidized glycan (aldehyde); or use of click chemistry (e.g., modification of nanoparticles with strained alkyne and linkage to an antibody modified with azide).

Any type of conjugation methods can be used for conjugating an antibody to a nanoparticle. Generally, to generate a desired yield of conjugate, an excess of antibody is provided in the conjugation reaction. This can result in a significant amount of free (unconjugated) antibody following the conjugation reaction. In some embodiments, there is also an amount of free unconjugated nanoparticles in the reaction mixture. The methods described herein are useful to purifying the conjugates from the free unconjugated members of the conjugation reaction. In some embodiments, a reagent is applied that will react with remaining reactive groups and prevent further reaction. As an example, conjugation between a maleimide-functionalized nanonparticle and a thiolated or reduced antibody will be stopped or quenched with an alkylating reagent including but not limited to N-ethylmaleimide. Reaction between an NHS-appended nanoparticle and a protein will be stopped or quenched with an amine including but not limited to ethanolamine

Once a conjugation has been performed, the resulting conjugation mixture (nanoparticle/antibody conjugate and unreacted free antibody and optionally free nanoparticle) is adjusted to have an ionic strength of at least 50 mM (e.g., between 50 -500mM, 50-300 mM, 100-300 mM, etc.). For example, the mixture's ionic strength can be adjusted with a high ionic strength buffer, e.g., a buffer containing a high concentration of ions to maintain the ionic strength listed above. In some embodiments, the buffer comprises at least 50 or 100 mM Na⁺or K. In some embodiments the buffer comprises phosphate (PO4³-). An exemplary buffer is phosphate buffered saline (PBS) (1X PBS = 10 mM sodium phosphate, 150 mM sodium chloride pH 7.8). In some embodiments, 0.5-3 X, e.g., 0.5-2.0 X, e.g., 1 X PBS is included in the mixture.

Ionic strength is calculated according to the following formula:

$$I = \frac{1}{2} {\sum }_{i = 1}^{n} {c}_{i} {z}_{i}^{2} ,$$

where cᵢ is the molar concentration of ion i (M, mol/L), zᵢ is the charge number of that ion, and the sum is taken over all ions in the solution.

Also included in the mixture is a sufficient amount of a surfactant to prevent aggregation and precipitation of the conjugates from the mixture, especially upon introduction of the high ionic strength buffer, which might otherwise result in aggregation or precipitation of the conjugates. In some embodiments, the surfactant is a nonionic polyalkylene glycol surfactant. In some embodiments, the surfactant is a polyoxypropylene-containing surfactant such as a poloxamer surfactant. Poloxamer surfactants are characterized by a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist that have slightly different properties. Poloxamer copolymers are commonly named with the letter “P” (for poloxamer) followed by three digits, the first two digits x 100 give the approximate molecular mass of the polyoxypropylene core, and the last digit x 10 gives the percentage polyoxyethylene content (e.g., P407 = Poloxamer with a polyoxypropylene molecular mass of 4,000 g/mol and a 70% polyoxyethylene content). For the Pluronic and Synperonic poloxamer tradenames, coding of these copolymers starts with a letter to define its physical form at room temperature (L = liquid, P = paste, F = flake (solid)) followed by two or three digits. The first digit (two digits in a three-digit number) in the numerical designation, multiplied by 300, indicates the approximate molecular weight of the hydrophobic chain; and the last digit x 10 gives the percentage polyoxyethylene content (e.g., F-68 indicates a polyoxypropylene molecular mass of 1,800 g/mol and a 80% polyoxyethylene content). Exemplary poloxamer surfactants include, but are not limited to, Pluronics F-68. The concentration of the surfactant used can be determined empirically (i.e., titrated such that precipitation of the conjugates does not occur). In some embodiments, the concentration of surfactant is 0.02%-1%, e.g., 0.05-0.2%, e.g., 0.1%.

The buffered high ionic strength mixture comprising the surfactant, conjugates, and free antibody are subsequently applied to a polysaccharide-based (i.e., comprising polysaccharides) size exclusion medium to separate the conjugate from free antibody and optionally from free nanoparticle. Size exclusion media when used in chromatography separates molecules based on their size or molecular weight. Size exclusion chromatography is based on the selective permeation of soluble proteins or other target molecules through a column of particles of a particular size, which particles have pores, typically, but not always, of a known size. Proteins of a size larger than the pores will not enter the pores. Large proteins that do not enter the pores pass around the particles and are eluted in the void volume (Vo). Very small proteins and salts are retained within the particles until the total permeation volume (Vt) is reached. Proteins that elute between the void volume and the total permeation volume are resolved, based upon the size and shape of their molecules.

In some embodiments, the polysaccharide in the polysaccharide-based size exclusion medium is agarose. In some embodiments, the polysaccharide is dextran. An exemplary agarose-based size exclusion medium is Superose 6 or 12 (available from GE Healthcare). In some cases, the medium will be provided in a column with the sample applied to the top of the chromatography and gravity forcing the sample through the column. In other embodiments, artificial pressure (e.g., HPLC) can also be applied as desired.

The output from the gel exclusion medium can be monitored for the presence of the conjugate, free antibody, or other components of the sample as desired to determine fractions that contain the conjugate and that are free, or at least have a reduced amount, of free antibody compared to the original conjugation mixture. In some embodiments, at least 90%, 95%, 99% of the untreated antibody in the conjugation reaction is removed in the resulting purified conjugate fractions. Exemplary methods for measuring output include monitoring a characteristic absorbance wavelength for the nanoparticle or antibody. The term “fraction” is used to refer to a portion of the output of chromatography and is not intended to limit how the output is collected or whether the output is collected in parts or continuously.

As an alternative to size-exclusion media, the high ionic strength, surfactant-containing conjugation mixture can be applied to a nanomembrane filter to separate the conjugates from free antibody. In these embodiments, the nanopores of the nanomembrane are selected to prevent passage of the conjugates while allowing for passage of the unconjugated antibody. The conjugation reaction can be passed through a nanoporous membrane such that conjugated nanoparticles will be retained and unconjugated antibodies will pass through the membrane. Repeated dilution and re-filtration will result in a preparation that is substantially free of unreacted antibody (e.g., at least 90%, 95%, 99% of the untreated antibody in the conjugation reaction is removed). The size of the nanopores will depend upon the size of the nanoparticle in the conjugate.

### EXAMPLE

An initial attempt to purify an antibody-nanopore conjugate from conjugation reactants such as free antibody was performed. High-resolution size exclusion chromatography on media with an appropriately high exclusion limit was attempted, however it was found that P-dot nanoparticles bound to many commercial media and would not elute from the columns. It was found that this binding was not significant on Superose 6 (GE Healthcare), Sephacryl 400, and Superdex 200.

Initial attempts to purify the conjugate on Sephacryl 400 and Superdex 200, however, were not successful. In these attempts, a nanoparticle-antibody conjugation sample in a low ionic strength 20 mM HEPES-KOH buffer was applied to 10 cm columns of Sephacryl 400 or Superdex 200. Separation of the conjugates from free antibody was poor (data not shown). P-dot was monitored by absorbance at 463 nm. IgG was monitored by densitometry on native gel.

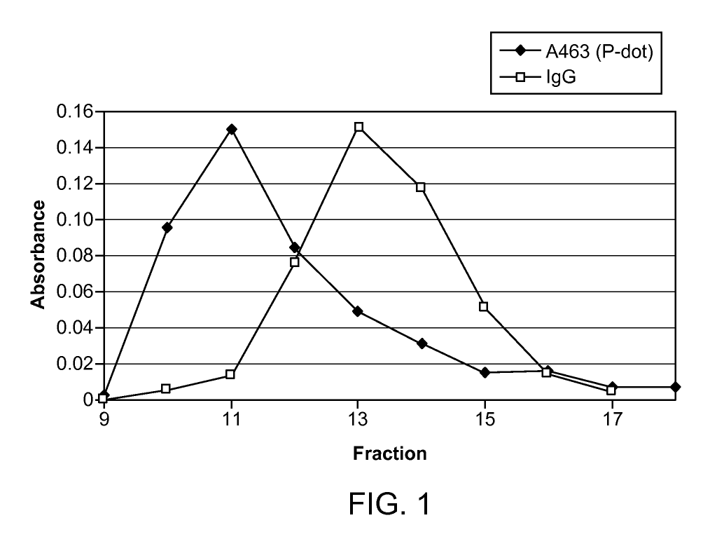

A 10 cm gravity column of Superose 6, which is an agarose-based size exclusion medium, was prepared. A nanoparticle-antibody conjugation sample in low ionic strength 20 mM HEPES-KOH buffer was added to the column, but did not result in helpful separation. The same experiment was repeated, but with a 30 cm column of Superose 6. While the peaks of the conjugate was separated from the free antibody, there was significant overlap of the shoulders of the peaks (see FIG. 1) and thus the conditions did not allow for optimal purification of the conjugate.

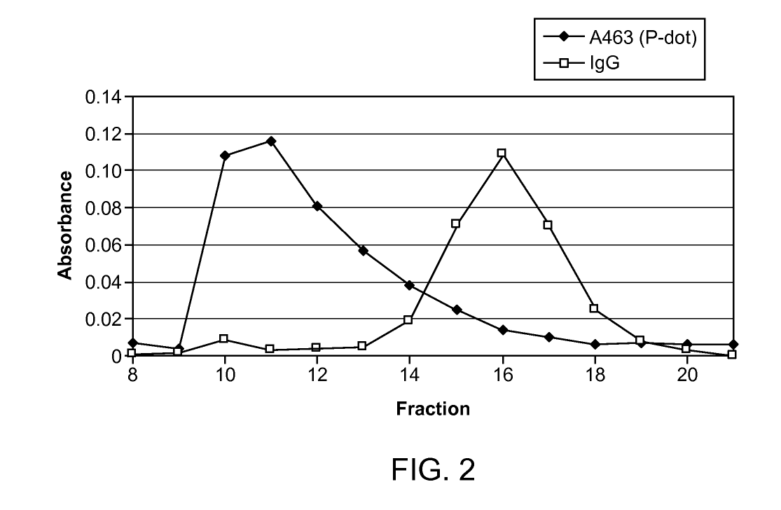

A higher ionic strength buffer (1 X PBS) was tested with the conjugate, but the higher ionic strength of the buffer resulted in precipitation of the conjugate, thereby preventing purification. A further mixture was prepared in 1 X PBS, but also including 0.1% Pluronic F-68. The conjugate remained in solution in this mixture and was applied to a 30 cm Superose 6 column. The resulting separation of the conjugate and free antibody (FIG. 2) was significantly better than the separation observed for the HEPES-KOH buffer mixture and allowed for purification of the antibody conjugates from free antibody.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method of purifying an antibody-nanoparticle conjugate from free antibody, the method comprising,
providing a mixture of the antibody-nanoparticle conjugate, the free antibody, a polyalkylene glycol surfactant, and a buffer, wherein the ionic strength of the mixture is at least 50 mM;
contacting the mixture to a polysaccharide-based size exclusion medium or nanomembrane filter to separate the antibody-nanoparticle conjugate from the free antibody; and
collecting fractions enriched for the antibody-nanoparticle conjugate from the medium or filter, thereby purifying the antibody-nanoparticle conjugate from the free antibody.

2. The method of claim 1, wherein the mixture is contacted to the polysaccharide-based size exclusion medium and the collecting comprises collecting fractions from the medium.

3. The method of claim 1, wherein the mixture is contacted to the nanomembrane filter and the collecting comprises collecting fractions from the filter.

4. The method of claim 2, wherein the polysaccharide-based size exclusion medium comprises agarose.

5. The method of claim 1, wherein the buffer comprises phosphate.

6. The method of claim 1, wherein the buffer is phosphate buffered saline (PBS).

7. The method of claim 6, wherein the PBS is at a concentration of 0.5-2.0 X.

8. The method of claim 1, wherein the antibody is an IgG antibody.

9. The method of claim 1, wherein the antibody is a tetrameric IgG antibody.

10. The method of claim 1, wherein the nanoparticle is a polymer dot (p-dot).

11. The method of claim 10, wherein the p-dot is 5-100 nm in diameter and is a colloidal semiconducting polymer.

12. The method of claim 10, wherein the p-dot is fluorescent.

13. The method of claim 1, wherein the surfactant is Pluronic F-68.

14. The method of claim 1, wherein the surfactant in the mixture is at a concentration of 0.02%-1.0%.

15. The method of claim 1, wherein the ionic strength of the mixture is 75-300 mM.

16. The method of claim 1, wherein the mixture further comprises free nanoparticle and the medium separates the free nanoparticle from the conjugate.
